(19) United States
(12) Patent Application Publication
(10) Pub. No.: US 2007/0254941 A1
(43) Pub. Date: Nov. 1, 2007

(54) Subtantially pure ropinirole hydrochloride, polymorphic form of ropinirole and process for their preparation

(75) Inventors: Bobba Venkata Siva Kumar, Navi Mumbai (IN); Sanjay Anantha Kale, Navi Mumbai (IN); Ajay Anant Audi, Thane (West) (IN); Nitin Sharad Chandra Pradhan, Thane (W) (IN)

Correspondence Address: M. CARMEN & ASSOCIATES, PLLC, 170 OLD COUNTRY ROAD, SUITE 400, MINEOLA, NY, 11501, US

(73) Assignee: Glenmark Pharmaceuticals Limited, Mumbai (IN)

(21) Appl. No.: 11/788,676

(22) Filed: Apr. 20, 2007

Related U.S. Application Data
(60) Provisional application No. 60/808,868, filed on May 26, 2006.

(30) Foreign Application Priority Data
Apr. 21, 2006 (IN) 638/MUM/2006
Jul. 19, 2006 (IN) 1154/MUM/2006

(51) Int. Cl. A61K 31/404; C07D 209/36

(52) U.S. Cl. 514/418; 548/484

(57) ABSTRACT

Ropinirole hydrochloride substantially free of impurities and a process for its preparation is provided. Also provided is ropinirole base substantially in polymorph Form A and a process for its preparation. Pharmaceutical compositions containing the same are also provided.

This application claims the benefit of U.S. Provisional Application No. 60/808,868, filed on May 26, 2006, and entitled “POLYMORPHIC FORM OF ROPINIROLE AND PROCESSES FOR THE PREPARATION THEREOF”, to Indian Provisional Application No. 638/MUM/2006, filed on Apr. 21, 2006, and entitled “POLYMORPHIC FORM OF ROPINIROLE AND PROCESSES FOR THE PREPARATION THEREOF”, and to Indian Provisional Application No. 1154/MUM/2006, filed on Jul. 19, 2006, and entitled “SUBTANTIALLY PURE ROPINIROLE HYDROCHLORIDE AND PROCESS FOR THE PREPARATION THEREOF”, the contents of each of which are incorporated by reference herein.

### BACKGROUND OF THE INVENTION

1. Technical Field

The present invention generally relates to a novel polymorphic form of ropinirole and a process for its preparation and substantially pure ropinirole hydrochloride having HPLC purity equal to or greater than 99.5%.

2. Description of the Related Art

Ropinirole, also known as 4-[2-(dipropylamino)ethyl]-1,3-dihydroindole-2-one, of formula (I) is used as an active constituent drug in the treatment of Parkinson. 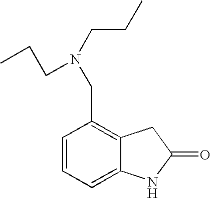
The hydrochloride salt of ropinirole is the form commercially sold under the trade name Requip®. Ropinirole hydrochloride is a non-ergoline dopamine agonist with high relative in vivo specificity and full intrinsic activity at the D₂ and D₃ dopamine receptors subtypes, binding with higher affinity to D₃ than to D₂ or D₄ receptor subtypes. Ropinirole has moderate in vitro affinity for opioid receptors. Ropinirole hydrochloride is indicated for the treatment of the signs and symptoms of idiopathic Parkinson's disease and for the treatment of moderate-to-severe primary Restless Legs Syndrome (RLS). See, e.g., The Merck Index, Thirteenth Edition, 2001, p. 1482-1483, monograph 8338; and Physician's Desk Reference, “Requip,” 58th Edition, p. 1604-1608 (2003). Ropinirole is a member of indolone class of potent non-ergot dopamine receptor antagonist marketed as a symptomatic treatment for Parkinson's disease. EP-0113964-B describes the use of such compounds as being useful in cardiovascular therapy.

U.S. Pat. No. 4,452,808 (“the '808 patent”) discloses ropinirole and its hydrochloride salt and a process for its preparation. One process disclosed in the '808 patent (see Example 2) generally involves (1) chlorinating and amidating 2-methyl-3-nitrophenyl acetic acid (II) to give 2-methyl-3-nitrophenyl-N,N-di-n-propyl acetamide (III), (2) reducing the acetamide (III) with borane in tetrahydrofuran (BH3-THF) to give 2-methyl-3-nitrophenylethyl-N,N-di-n-propyl amine (IV), (3) reacting the amine (IV) with diethyl oxalate to provide ethyl-6-(2-di-n-popylaminoethyl)-2-nitrophenylpyruvate (V), (4) hydrolyzing and decarboxylating the nitrophenylpyruvate (V) to give 2-nitro-6-(2-di-n-propylaminoethyl)phenyl acetic acid hydrochloride, and (5) reductive cyclizing the acetic acid hydrochloride to give ropinirole hydrochloride (I). Example 1 isolates ropinirole hydrochloride as a pale yellow crystalline solid whereas Example 2 isolates as a white residue which is then crystallized with very large volumes of acetonitrile.

U.S. Pat. No. 4,997,954 discloses a process for the preparation of ropinirole hydrochloride of 97% HPLC purity which, upon purification by crystallization or basification and acidification, yields pure Ropinirole HCl having 98-99% purity.

U.S. Pat. No. 5,336,781 discloses a process for the preparation of off white solids containing impurity which imposes high colour index to the product.

WO 2005/080333 discloses a process for purifying ropinirole hydrochloride and polymorphic Form I of ropinirole hydrochloride.

WO 2005/074387 discloses crystalline form I and form II of ropinirole hydrochloride and process for the preparation of these forms.

WO 2005/105741 discloses substantially pure ropinirole hydrochloride having HPLC purity equal to or greater than 99.5%.

The inventors of the present invention found the presence of despropyl indolone impurity, N-hydroxy indolone impurity and 5-ethyl indolone impurities in the final product. Efforts are made to prepare pharmaceutical products of a high grade and with a minimum amount of impurities present. The control of impurities requires a study of various options to decide upon the reaction conditions and testing protocols necessary to insure that drugs which are administered to the public are substantially pure. Accordingly, there remains a need for an improved process for preparing ropinirole hydrochloride that eliminates or substantially reduces the impurities in a convenient and cost efficient manner to provide substantially pure of ropinirole hydrochloride.

Polymorphism is the occurrence of different crystalline forms of a single compound and it is a property of some compounds and complexes. Thus, polymorphs are distinct solids sharing the same molecular formula, yet each polymorph may have distinct physical properties. Therefore, a single compound may give rise to a variety of polymorphic forms where each form has different and distinct physical properties, such as different solubility profiles, different melting point temperatures and/or different x-ray diffraction peaks. Since the solubility of each polymorph may vary, identifying the existence of pharmaceutical polymorphs is essential for providing pharmaceuticals with predictable solubility profiles. It is desirable to investigate all solid state forms of a drug, including all polymorphic forms, and to determine the stability, dissolution and flow properties of each polymorphic form. Polymorphic forms of a compound can be distinguished in a laboratory by X-ray diffraction spectroscopy and by other methods such as, infrared spectrometry. Additionally, polymorphic forms of the same drug substance or active pharmaceutical ingredient, can be administered by itself or formulated as a drug product (also known as the final or finished dosage form), and are well known in the pharmaceutical art to affect, for example, the solubility, stability, flowability, tractability and compressibility of drug substances and the safety and efficacy of drug products.

The discovery of new polymorphic forms of a pharmaceutically useful compound provides a new opportunity to improve the performance characteristics of a pharmaceutical product. It also adds to the material that a formulation scientist has available for designing, for example, a pharmaceutical dosage form of a drug with a targeted release profile or other desired characteristic. A new polymorphic form of ropinirole base has now been discovered.

### SUMMARY OF THE INVENTION

In accordance with one embodiment of the present invention, substantially pure ropinirole hydrochloride is provided having a HPLC purity equal to or greater than about 99.5% and a content of despropyl indolone impurity, N-hydroxy indolone impurity and 5-ethylindolone impurity of less than about 0.15%.

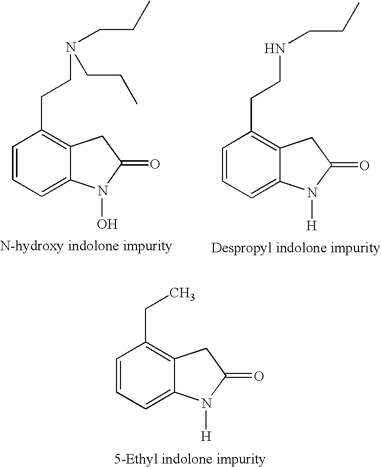

In accordance with a second embodiment of the present invention, a process for the preparation of substantially pure ropinirole hydrochloride is provided comprising:
(a) providing a solution comprising ropinirole base of Formula II in one or more organic solvents; 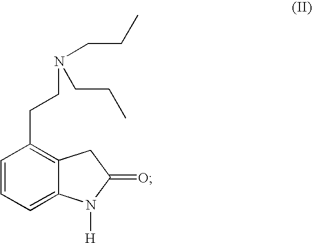
(b) extracting the solution with a base at a pH of about 10 to about 12;
(c) extracting the solution with an acid;
(d) substantially removing the solvent from the solution;
(e) dissolving the resulting solids in one or more polar solvents;
(f) adding hydrochloric acid under substantially dry conditions; and
(g) recovering the substantially pure ropinirole hydrochloride.

The advantages of the process of the present invention include at least:
1. All existing methods for the preparation of ropinirole hydrochloride require extra steps of purification for the removal above specified impurities. This may lead to higher costs and cause a drastic load on the effluent treatment system.
2. The present invention incorporates the use of very economical and environmentally friendly reagents like sodium hydroxide and hydrochloric acid to remove the above-specified impurities. Such improvements make the process easier and friendlier on a commercial scale.

In accordance with a third embodiment of the present invention, a process for the preparation of ropinirole substantially in polymorph Form A is provided comprising:
(a) treating crude ropinirole base or a salt thereof with an inorganic base in a solvent; and
(b) recovering the ropinirole substantially in polymorphic Form A.

In accordance with a fourth embodiment of the present invention, ropinirole substantially in polymorph Form A is provided.

In accordance with a fifth embodiment of the present invention, ropinirole substantially in the polymorph Form A and having a powder X-ray diffraction (XRD) pattern substantially in accordance with FIG. 1 is provided.

In accordance with a sixth embodiment of the present invention, ropinirole substantially in the polymorph Form A is provided exhibiting a characteristic peak (expressed in degrees 2θ±0.2°θ) at approximately 6.81.

In accordance with a seventh embodiment of the present invention, ropinirole substantially in the polymorph Form A and having an infra-red (IR) spectrum pattern substantially in accordance with FIG. 2 is provided.

In accordance with an eighth embodiment of the present invention, ropinirole substantially in the polymorphic Form A is provided exhibiting characteristic (IR) peaks (expressed in cm⁻¹) at approximately one or more of the positions: 3420, 2968, 2937, 2878, 2644, 1698, 1616, 1460, 1384, 1351, 1325, 1290, 1252, 1161, 1083, 967, 884,839, 800, 705, 662, 559.

In accordance with a ninth embodiment of the present invention, a pharmaceutical composition is provided comprising a therapeutically effective amount of ropinirole substantially in the polymorphic Form A.

### BRIEF DESCRIPTION OF THE DRAWINGS

FIG. 1 is a characteristic X-ray powder diffraction pattern of polymorph Form A of ropinirole.

FIG. 2 is a characteristic Infra-Red (IR) spectrum of polymorph Form A of ropinirole.

### DETAILED DESCRIPTION OF THE INVENTION

The present invention provides substantially pure ropinirole hydrochloride having HPLC purity equal to or greater than about 99.5%. The present invention also provides substantially pure ropinirole hydrochloride having HPLC purity equal to or greater than about 99.5% and content of impurities like despropyl indolone impurity, N-hydroxy indolone impurity and 5-ethyl indolone impurity of less than about 0.15%, preferably less than about 0.1%.

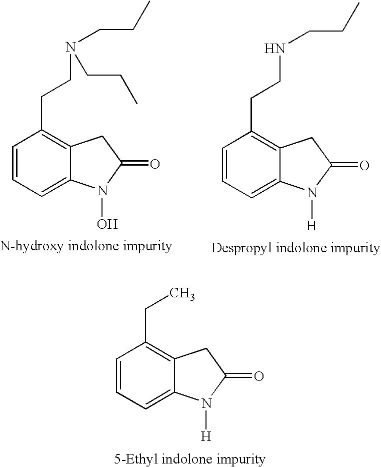

Furthermore, the present invention provides a process for the preparation of substantially pure ropinirole hydrochloride comprising:
(a) providing a solution comprising ropinirole base of Formula II in one or more organic solvents; 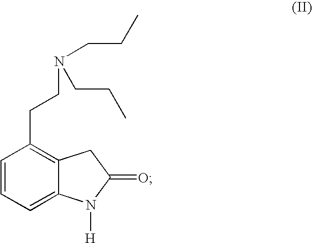
(b) extracting the solution with a base at a pH of about 10 to about 12;
(c) extracting the solution with an acid;
(d) substantially removing the solvent from the solution;
(e) dissolving the resulting solids in one or more polar solvents;
(f) adding hydrochloric acid under substantially dry conditions; and
(g) recovering the substantially pure ropinirole hydrochloride.

The solution of ropinirole may be provided by dissolving any form of ropinirole in a suitable solvent or obtaining an existing solution from a previous processing step. The organic solvents include, but are not limited to, benzene, alkyl, aryl, or halo substituted benzenes, chlorinated olefins such as dichloro methane, dichloro ethane or cyclic and acyclic hydrocarbons such as n-pentane, n-hexane and n-octane, cyclic and acyclic ethers such as diethyl ether, tetrahydrofuran, and dioxane and esters such as ethyl acetate, methyl acetate, isopropyl acetate, butyl acetate and the like and mixtures thereof.

The dissolution can be carried out at a temperature ranging from about 20° C. to about 80° C. and preferably at room temperature. The suitable bases include, but are not limited to, alkali and alkaline earth metal hydroxides, preferably sodium hydroxide, potassium hydroxide, and barium hydroxide. Removal of solvent is accomplished by, for example, substantially complete evaporation of the solvent, concentrating the solution. Alternatively, the solvent may also be removed by evaporation.

The polar solvents include, but are not limited to, alcohols, esters, nitrile, ethers and polar aprotic solvents such as DMF, DMSO, N, N-DMA, and NMP. The hydrochloric acid can be added in substantially anhydrous conditions at temperature from about 0° C. to about 100° C., preferably at a temperature from about 0° C. to about 50° C., and more preferably at temperature from about 0° C. to about 30° C. Substantially pure ropinirole hydrochloride can be isolated by filtration, decanting or any other isolation technique known to the skilled in the art.

The ropinirole base obtained from the reductive cyclization of 2-substituted-o-nitro phenyl acetic acid derivative may be dissolved with one or more aromatic solvents. A clear solution thus obtained can be treated with dilute aqueous alkali metal hydroxides having strengths ranging from about 2% to about 30%; and preferably from about 15% to about 20% at a pH of about 11 to about 12 and at a temperature ranging from about 0° C. to about 80° C.; and preferably from about 20° C. to about 30° C.

The organic layer containing ropinirole free base may further be treated with dilute aqueous inorganic acids having strengths ranging from about 1% to about 10%, preferably from about 0.5% to about 2% at a pH of about 7 to about 7.5 at a temperature ranging from about 0° C. to about 50° C., preferably from about 20° C. to about 30° C. The resulting organic layer containing ropinirole free base may further be concentrated under reduced pressure at temperature ranging from about 30° C. to about 100° C., and preferably from about 30° C. to about 50° C. to obtain ropinirole base. The resulting solid can then be dissolved in one or more polar solvents such as water, aliphatic alcohols like methanol, ethanol, and isopropanol, aliphatic ketones such as acetone, and ethyl methyl ketone, and aliphatic cyanides such as acetonitrile, aliphatic acids such as acetic acid, and propionic acid, esters such as methyl acetate, ethyl acetate, and isopropyl acetate and the like and mixtures thereof at temperatures ranging from about 20° C. to about 150° C. Preferably the solvent used for the dissolution of ropinirole free base is acetonitrile.

The solution can then be cooled to about 0° C. to about 20° C., followed by the addition of hydrochloric acid in gaseous or solution form to achieve a pH of about 1 to about 2 to recover substantially pure ropinirole hydrochloride having purity equal to or more than about 99.5%. Generally crude ropinirole hydrochloride may be dissolved in one or more solvents at temperature ranging from about 10° C. to about 70° C., preferably from about 60° C. to about 65° C. The resulting clear solution of ropinirole hydrochloride can be further cooled to a temperature of about 0° C. to about 30° C., and preferably from about 0° C. to about 15° C., to afford pure ropinirole hydrochloride having HPLC purity greater than about 99.5%.

The substantially pure ropinirole hydrochloride can be recovered after removing the solvent by performing distillation, distillation under vacuum and filtration under vacuum, decantation, and centrifugation. The product obtained may additionally be dried, for example in a tray drier, or dried under vacuum and/or in a Fluid Bed Drier, to achieve the desired residual solvent values.

Preferable solvents include alkanols (e.g. methanol, ethanol, isopropanol etc.), ketonic solvents (e.g. acetone, methyl ethyl ketone, methyl isobutyl ketone), cyclic and acyclic ethers (e.g. diethyl ether ,tetrahydrofuran, dioxane etc.), esters (e.g. ethyl acetate isopropyl acetate, methyl acetate), aromatic solvents (e.g. benzene or alkyl, aryl, halo substituted benzenes), chlorinated olefins (e.g. dichloro methane, dichloro ethane), cyclic and acyclic hydrocarbons (e.g. n-pentane, n-hexane n-octane etc.), and the like and mixtures thereof. The preferred solvent mixture is toluene and methanol.

The term “ropinirole hydrochloride substantially free of despropyl indolone, N-hydroxy indolone and 5-Ethyl indolone” as used herein shall be understood to mean ropinirole hydrochloride formed with little to no content of despropyl indolone, N-hydroxy indolone and 5-ethyl indolone. In this manner, the amount of despropyl indolone, N-hydroxy indolone and 5-Ethyl indolone, if present, resulting from the process for preparing ropinirole hydrochloride present will be in relatively minor amounts, e.g., less than about 0.15% weight percent, preferably less than about 0.1 weight percent and most preferably 0 weight percent.

The present invention is also directed to a novel polymorphic form of ropinirole, designated Form A. Ropinirole substantially in polymorph Form A is a particularly useful intermediate in preparing pharmaceutically acceptable salts thereof, e.g., ropinirole hydrochloride, and polymorphs thereof. In general a process for preparing ropinirole substantially in polymorph Form A includes at least (a) treating crude ropinirole base or a salt thereof with an inorganic base in a solvent; and (b) recovering the ropinirole substantially in polymorph Form A. In step (a) of the process, ropinirole base or a salt thereof, e.g., hydrochloride salt, is dissolved in a suitable solvent to prepare a solution. Suitable solvents for use herein include, but are not limited to, water, water-miscible solvents such as methanol, ethanol, isopropanol, t-butanol, acetonitrile, 1,4-dioxane and the like, and water-immiscible solvents such as esters, ethyl acetate, ethyl acetoacetate, methylene chloride, ethylene chloride, chloroform, methyl tert butyl ether, toluene and the like and mixtures thereof. The preferred solvent is water.

The ropinirole base or a salt thereof should be mixed with the solvent for a time period ordinarily ranging about 30 to about 40 minutes to form a solution. An inorganic base is then added to the solution, which basifies the solution to a pH of about 8 to about 9. The inorganic base used herein can be, for example, aqueous or solid alkaline earth metal hydroxides such as sodium hydroxide, potassium hydroxide, barium hydroxide, calcium hydroxide and the like, alkali or alkaline earth metal carbonates such as sodium bicarbonate, potassium carbonate, calcium carbonate, barium carbonate and the like and mixtures thereof. The preferred inorganic base used herein is sodium bicarbonate. The temperature of the reaction mixture may range from about 0° C. to about 50° C., preferably from about 20° C. to about 30° C.

In step (b) of the process, ropinirole substantially in polymorph Form A is recovered from the solution. The resulting polymorph may then be recovered by techniques well known in the art, e.g., filtration, centrifugation, decanting, etc. The filtered solid may then be washed and dried to produce crystals of Form A of ropinirole.

The present invention also provides ropinirole substantially in polymorph Form A. Crystallinity of the novel polymorph of this invention may be measured using methods familiar to those skilled in the art. The novel polymorph of the present invention as described herein below was characterized by X-ray powder diffraction (XRD) and IR analysis. The X-Ray powder diffraction spectrum for the novel polymorph was measured by an X-ray powder Diffractometer equipped with a Cu-anode (λ=1.54 Angstrom), X-ray source operated at 45 kV, 40 mA and a Ni filter is used to strip K-beta radiation. Two-theta calibration is performed using an NIST SRM 640c Si standard. The sample was analyzed using the following instrument parameters: measuring range=2-50° 2θ; step width=0.017°; and measuring time per step=5 sec. The Infra-Red (“IR”) spectrum of polymorph Form A of ropinirole was obtained on a Perkin Elmer FT-IR spectrometer. The sample was prepared by KBr powder technique registering the spectrum on reflectance.

The ropinirole substantially in polymorphic Form A of the present invention exhibits characteristic peaks (expressed in degrees 2θ±0.2°θ) at approximately one or more of the positions of Table 1.

TABLE 1

| 2θ | d-spacing | Relative Intensity |
|---|---|---|
| (degrees) | (\acute{Å}) | (%) |
| 6.81 | 12.96 | 100 |
| 11.93 | 7.41 | 15.56 |
| 12.11 | 7.3 | 2.12 |
| 13.28 | 6.66 | 5.5 |
| 13.49 | 6.56 | 8.09 |
| 15.5 | 5.71 | 22.91 |
| 15.6 | 5.67 | 10.28 |
| 16.8 | 5.27 | 14.33 |
| 18.37 | 4.829 | 11.87 |
| 20.5 | 4.33 | 25.45 |
| 20.85 | 4.26 | 4.55 |
| 21.23 | 4.18 | 6.38 |
| 21.65 | 4.1 | 2.93 |
| 22.92 | 3.88 | 29.16 |
| 23.4 | 3.8 | 8.34 |
| 23.62 | 3.76 | 9.99 |
| 23.94 | 3.71 | 2.99 |
| 26.43 | 3.37 | 6.26 |
| 26.66 | 3.34 | 5.1 |
| 27.33 | 3.26 | 5.69 |
| 28.4 | 3.14 | 4.01 |
| 29.11 | 3.06 | 1.03 |
| 29.85 | 2.99 | 1.99 |
| 30.088 | 2.97 | 2.17 |
| 31.28 | 2.859 | 2.45 |
| 32.72 | 2.73 | 1.14 |
| 34.48 | 2.6 | 1.4 |
| 37.87 | 2.37 | 1.46 |
| 38.32 | 2.348 | 1.43 |
| 38.88 | 2.31 | 1.29 |

The ropinirole substantially in polymorph Form A of the present invention also exhibits characteristic (IR) peaks (expressed in cm⁻¹) at approximately one or more of the positions: about 3420, 2968, 2937, 2878, 2644, 1698, 1616, 1460, 1384, 1351, 1325, 1290, 1252, 1161, 1083, 967, 884,839, 800, 705, 662, 559.

In another aspect of the present invention provides ropinirole substantially in polymorph Form A, having a chemical purity of about 96% or more as measured by HPLC. , preferably about 99% or more, more preferably about 99.5% or more.

Another aspect of the present invention is directed to a pharmaceutical composition containing ropinirole substantially in polymorph Form A and/or ropinirole hydrochloride substantially free of despropyl indolone, N-hydroxy indolone and 5-Ethyl indolone, wherein the D₅₀ and D₉₀ particle size of the unformulated ropinirole or pharmaceutically acceptable salt thereof used as starting material is less than about 400 microns, preferably less than about 200 microns, more preferably less than about 150 microns, still more preferably less than about 50 microns and most preferably less than about 15 microns. Any milling, grinding micronizing or other particle size reduction method known in the art can be used to achieve desired particle size range set forth above.

The ropinirole substantially in polymorph Form A and/or substantially pure ropinirole or pharmaceutically acceptable salts thereof free of despropyl indolone, N-hydroxy indolone and 5-ethyl indolone thus obtained may then be formulated into a pharmaceutical composition or dosage form. Such compositions and dosage forms include, for example, compacted tablets, powder suspensions, capsules, and the like. The compositions of the present invention can be administered to humans and animals either orally, rectally, parenterally (intravenous, intramuscular, or subcutaneous), intracistemally, intravaginally, intraperitoneally, locally (powders, ointments or drops), or as a buccal or nasal spray. For example, the active ingredient of the invention, or salts or solvates thereof can be administered orally, buccally or sublingually in the form of tablets, capsules (including soft gel capsules), ovules, elixirs, solutions or suspensions, which may contain flavoring or coloring agents, for immediate-, delayed-, modified-, or controlled-release such as sustained-, dual-, or pulsatile delivery applications. The ropinirole substantially in polymorph Form A and/or substantially pure ropinirole or pharmaceutically acceptable salts thereof disclosed herein also may be administered as suppositories, ophthalmic ointments and suspensions, and parenteral suspensions, which are administered by other routes. The most preferred route of administration is oral.

The active ingredient of the invention may also be administered via fast dispersing or fast dissolving dosage forms or in the form of high energy dispersion or as coated particles. Suitable pharmaceutical composition of the invention may be in coated or un-coated form as desired.

Tabletting compositions may have few or many components depending upon the tabletting method used, the release rate desired and other factors. For example, the compositions of the present invention may contain diluents such as cellulose-derived materials (e.g. powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses), starch, pregelatinized starch, inorganic diluents (e.g. calcium carbonate and calcium diphosphate) and other diluents known to one of ordinary skill in the art. Further suitable diluents include waxes, sugars (e.g. lactose) and sugar alcohols (e.g. mannitol and sorbitol), acrylate polymers and copolymers, as well as pectin, dextrin and gelatin.

Other excipients contemplated by the present invention include binders (e.g. acacia gum, pregelatinized starch, sodium alginate, glucose and other binders used in wet and dry granulation and direct compression tableting processes), disintegrants (e.g. sodium starch glycolate, crospovidone, low-substituted hydroxypropyl cellulose and others), lubricants (e.g. magnesium and calcium stearate and sodium stearyl fumarate), flavorings, sweeteners, preservatives, pharmaceutically acceptable dyes and glidants (e.g. silicon dioxide).

Capsule dosages will contain the solid composition within a capsule which may be coated with gelatin. Tablets and powders may also be coated with an enteric coating. The enteric-coated powder forms may have coatings comprising phthalic acid cellulose acetate, hydroxypropylmethyl cellulose phthalate, polyvinyl alcohol phthalate, carboxymethylethylcellulose, a copolymer of styrene and maleic acid, a copolymer of methacrylic acid and methyl methacrylate, and like materials. Optionally, the capsule dosage may be formulated with suitable plasticizers and/or extending agents. A coated tablet may have a coating on the surface of the tablet or may be a tablet comprising a powder or granules with an enteric coating.

Actual dosage levels of the substantially pure ropinirole in the compositions of the invention may be varied to obtain an amount of that is effective to obtain a desired therapeutic response for a particular composition and method of administration. The selected dosage level therefore depends upon such factors as, for example, the desired therapeutic effect, the route of administration, the desired duration of treatment, and other factors. The total daily dose of the compounds of this invention administered to a host in single or divided dose and can vary widely depending upon a variety of factors including, for example, the body weight, general health, sex, diet, time and route of administration, rates of absorption and excretion, combination with other drugs, the severity of the particular condition being treated, etc. The pharmaceutical compositions herein can be formulated in any release form, e.g., immediate release, sustained release, controlled release, etc.

The following examples are provided to enable one skilled in the art to practice the invention and are merely illustrative of the invention. The examples should not be read as limiting the scope of the invention.

### EXAMPLE 1

### Preparation of Pure Ropinirole Hydrochloride

Ropinirole base (308.0 gram) was dissolved in 4.5 liter of toluene. This was followed by addition of 7.5 liter of 15-20% aq. NaOH solution at 20-35° C. The solution was stirred vigorously at 22-25° C. The toluene layer was separated and extracted with 12 lit 10% aq. NaOH solution at 22-25° C. The toluene layer was extracted twice with 12 liter of 10% aq. HCl solution at 22-25° C. The organic layer was then washed twice with 6 liter water. The toluene layer was concentrated under reduced pressure at 50-65° C. The resulted gummy solid was dissolved in 1.2 liter of acetonitrile. The clear solution was then slowly cooled to room temperature and then further cooled to 20° C. This was followed by slowly addition of 10-25% IPA-HCl solution (308 ml) to the reaction mass at same temperature to get a slurry of precipitated ropinirole hydrochloride. The slurry was then stirred at temperature of 15-20° C. for 1 hour followed by filtration and washing with 600 ml of acetonitrile. The resulting solid was further dried at 50-60° C. under vacuum to afford pure ropinirole hydrochloride (155 grams,) HPLC. purity 99.86%

Table 2 provides purity and impurity data (HPLC) of three batches of ropinirole hydrochloride prepared by the process of present invention.

TABLE 2

| Sr. No. | Purity | A | B | C |
|---|---|---|---|---|
| 1 | 99.80% | BDL % | 0.07% | BDL % |
| 2 | 99.75% | BDL % | 0.07% | BDL % |
| 3 | 99.74% | 0.01% | 0.08% | BDL % |

BDL: below detection level
A - 4-[2-(n-propylamino)ethyl]-1,3-dihydro-2H-indol-2-one
B - 4-(2-(dipropylamino)ethyl)-1-hydroxyindolin-2-one
C - 4-ethyl-1,3-dihydro-2H-indol-2-one

### EXAMPLE 2

### Preparation of Substantially Pure Ropinirole Hydrochloride

Into a four-neck 500 ml flask equipped with a mechanical stirring condenser and thermometer was charged with 700 ml toluene and 100 gram crude ropinirole hydrochloride. the supsension was slowly heated at 65 to 75° C., followed by slow addition of 350-400 ml of methanol to get clear solution. The solution was then stirred at the same temperature for 1 hour followed by filtration to remove insoluble material. The clear filtrate was then slowly cooled to room temperature and then further cooled to 5-15° C. and stirred for 1 hour. The resulting solid was filtered and washed with 100 ml toluene/methanol mixture (8:2) to afford substantially pure ropinirole hydrochloride, which was further dried at 50-60° C. Yield: 88 grams, HPLC purity 99.5%.

### EXAMPLE 3

### Preparation of Ropinirole Free Base

Isopropanol (5000 ml) and 2-nitro-6-N,N-di-n-propyl-phenyl acetic acid (473 g) were added in a round bottom flask. Slurry of 10% Pd-carbon in isopropanol (500 ml) was added under nitrogen in one lot at a temperature ranging from about 20° C. to about 30° C. The temperature was slowly raised to a range of about 70° C. to about 75° C. Ammonium formate (393 g) was added in four lots at a temperature ranging from about 70° C. to about 75° C. The reaction mixture was stirred for about 2 hours at a temperature ranging from about 75° C. to about 80° C. After completion of reaction, the reaction mixture was cooled to a temperature of about 60° C.

The catalyst was filtered and washed with isopropanol (500 ml). Isopropanol was then distilled out under reduced pressure below a temperature of about 50° C. Toluene (1400 ml) was added to the reaction mixture at a temperature ranging from about 40° C. to about 50° C. The reaction mixture was cooled to a temperature ranging from about 20° C. to about 30° C. Water (1 ml) was added to the reaction mixture, stirred, and the layers were allowed to settle. The upper organic layer was separated. The upper toluene layer was washed twice with 4-5% aqueous NaOH solution (2×900 ml), followed with water (900 ml).

The pH of the toluene layer was adjusted to a range of about 7 to about 7.5 with 0.1-1% HCl solution. The organic layer was separated and the pH was adjusted to a range of about 1 to about 1.5 with 1.5-2.5% HCl solution. The aqueous layer was taken and the pH was adjusted to a range of about 8 to about 8.5 with sodium bicarbonate. The reaction mixture was stirred for about 1 hour at a room temperature (a temperature ranging from about 25° C. to about 30° C.).

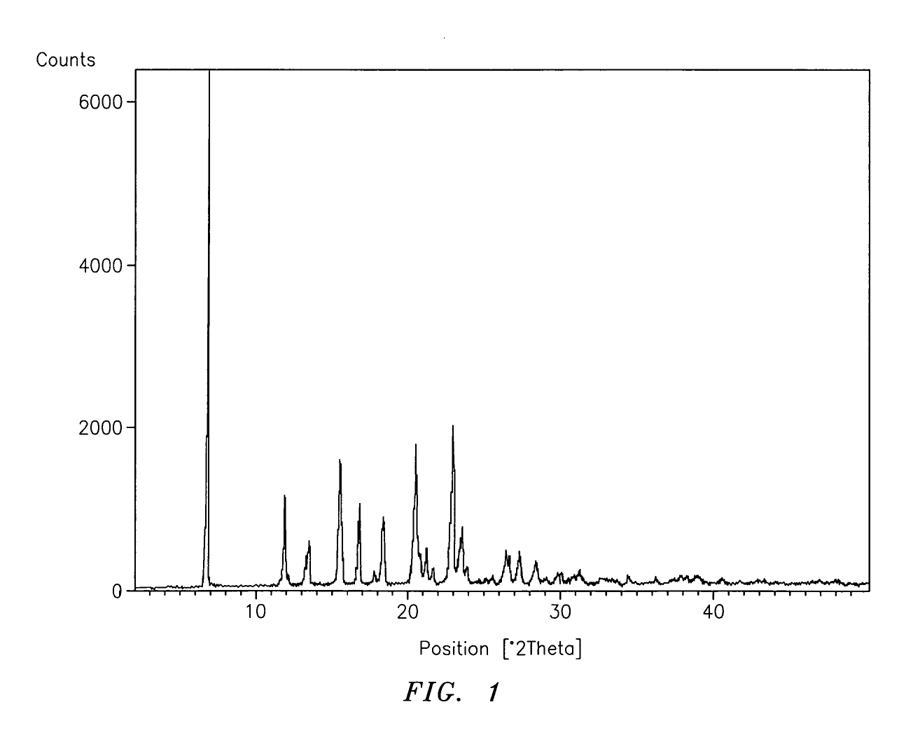

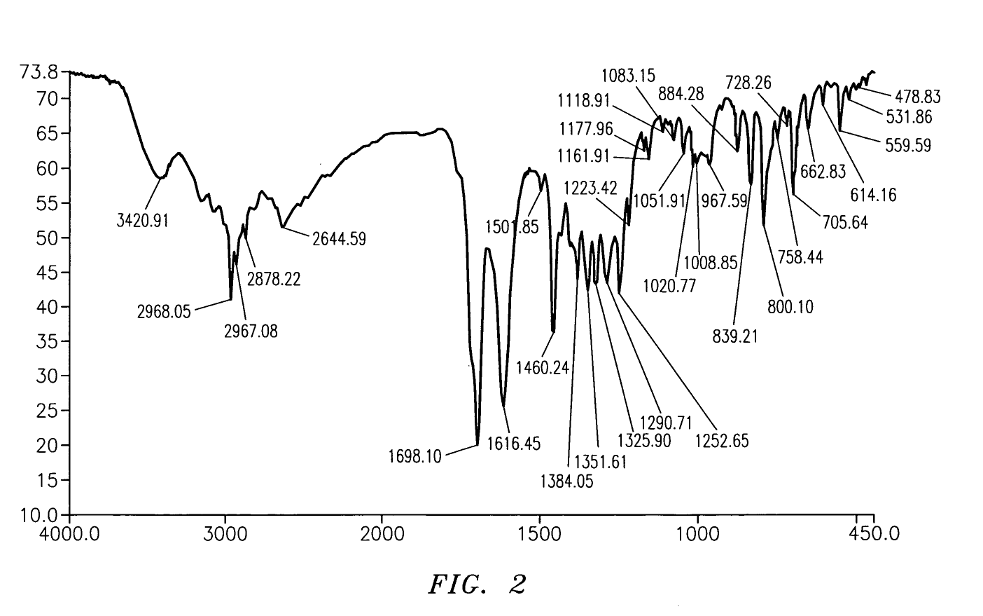

The solids obtained were filtered, washed with chilled water (215 ml) and acetonitrile (215 ml). The solids that were yielded from this were dried by vacuum to produce ropinirole free base (about 225-230 g) with HPLC. purity equal to or more than 99%. A small sample of this was analyzed for powder XRD and IR as shown in FIGS. 1 and 2.

Powder XRD—(2θ)—6.81, 11.93, 12.11, 13.26, 13.49, 15.5, 15.6, 16.8, 18.37, 20.5, 20.85, 21.23, 21.65, 22.92, 23.4, 23.6, 23.94, 26.43, 26.66, 27.33, 28.4, 29.11, 29.85, 30.09, 31.28, 32.72, 34.48, 37.87, 38.32, 38.88.

IR—(cm⁻¹)—3420, 2968, 2937, 2878, 2644, 1698, 1616, 1460, 1384, 1351, 1325, 1290, 1252, 1161, 1083, 967, 884, 839, 800, 705, 662, 559

### EXAMPLE 4

### Preparation of Ropinirole Free Base

Isopropanol (5000 ml) and 2-nitro-6-N,N-di-n-propyl-phenyl acetic acid (473 g) were added in a round bottom flask. A slurry of 10% Pd-carbon in isopropanol (500 ml) was added under nitrogen in one lot at a temperature ranging from about 20° C. to about 30° C. The temperature of the reaction mixture was slowly raised to a range of about 50° C. to about 60° C. Tri ethyl ammonium formate (903 g) was added to the reaction mixture over a period of about 15 minutes to about 30 minutes at a temperature ranging from about 60° C. to about 65° C. The reaction mixture was stirred for about 2 hours at a temperature ranging from about 60° C. to about 65° C. After completion of the reaction, the reaction mixture was cooled to a temperature of about 60° C.

The catalyst was filtered and washed with isopropanol (500 ml). Isopropanol was then distilled out under reduced pressure below a temperature of about 50° C. Toluene (1400 ml) was added to the reaction mixture at a temperature ranging from about 40° C. to about 50° C. The reaction mixture was cooled to a temperature ranging from about 20° C. to about 30° C. Water (1000 ml) was added to the reaction mixture, stirred, and the layers were allowed to settle. The upper organic layer was separated. The upper toluene layer was washed twice with 4-5% aqueous NaOH solution (2×900 ml), followed with water (900 ml).

The pH of the toluene layer was adjusted to a range of about 7 to about 7.5 with 0.1-1% HCl solution. The organic layer was separated and the pH was adjusted to a range of about 1 to about 1.5 with 1.5-2.5% HCl solution. The aqueous layer was taken and the pH was adjusted to a range of about 8 to about 8.5 with sodium bicarbonate. The reaction mixture was stirred for about 1 hour at a room temperature (a temperature ranging from about 25° C. to about 30° C.). The solids obtained were filtered, washed with chilled water (215 ml) and acetonitrile (215 ml). The solids were then dried under a vacuum to produce ropinirole free base (about 290-300 g) with HPLC purity equal to or more than 99%.

### EXAMPLE 5

### Preparation of Ropinirole HCl from Ropinirole Free Base

The solid obtained in Example 1 was taken without further drying and purified in acetonitrile (1700 ml). The reaction mixture was then cooled to a temperature ranging from about 5° C. to about 10° C. A 20% isopropanol/HCl solution was added at a temperature ranging from about 10° C. to about 15° C. The reaction mixture was stirred for about 7 hours. The solids were filtered, washed with chilled acetonitrile (30 ml) and dried to give crude ropinirole hydrochloride. The crude ropinirole hydrochloride was taken in toluene (4.5 lit.). To the suspension was added 7.5 lit of 15-20% aq. NaOH solution at 22-25° C. The solution is stirred vigorously at 22-25° C. The toluene layer was separated and extracted with 12 liter 10 aq. NaOH solution at 22-25° C.

The toluene layer was than washed twice with 6 liters of water. The toluene layer was extracted twice with 12 liters, 1% aq. HCl solution at 22-25° C. The organic layer was then washed twice with 6 liters of water. The toluene layer was concentrated under reduced pressure to a temperature ranging from about 50° C. to about 65° C. The residual gummy solids were dissolved in acetonitrile (1.2 lit) The clear solution was then slowly cooled to room temperature and then further cooled to 20° C. 10-25% IPA-HCl solution (308 ml) was then added to the reaction mass slowly at the same temperature to get slurry of precipitated ropinirole hydrochloride. The suspension obtained was then stirred at temperature of 15-20° C. for 1 hour and filtered off the solids on a Buchner funnel and washed twice with acetonitrile (600 ml). The filtered product was dried at 50-60° C. under vacuum until the LOD was less than 0.5% to provide pure ropinirole hydrochloride (150 g) HPLC purity more than 99.5%

While the above description contains many specifics, these specifics should not be construed as limitations of the invention, but merely as exemplifications of preferred embodiments thereof. Those skilled in the art will envision many other embodiments within the scope and spirit of the invention as defined by the features and advantages appended hereto.

What is claimed is:

1. Ropinirole base substantially in polymorph form A.

2. The ropinirole base substantially in polymorph form A of claim 1, characterized by having at least one of the following characteristics:
(a) an X-ray diffraction (XRD) pattern substantially in accordance with FIG. 1; and/or
(b) a Infra-Red (IR) spectrum substantially in accordance FIG. 2.

3. The ropinirole base substantially in polymorph form A of claim 1, exhibiting a characteristic peak (expressed in degrees 2θ±0.2°θ) at about 6.81.

4. The ropinirole base substantially in polymorph form A of claim 1, exhibiting characteristic peaks (expressed in cm⁻¹) at approximately about one or more of the positions: 3420, 2968, 2937, 2878, 2644, 1698, 1616, 1460, 1384, 1351, 1325, 1290, 1252, 1161, 1083, 967, 884, 839, 800, 705, 662 and 559.

5. A pharmaceutical composition comprising a therapeutically effective amount of the ropinirole base substantially in polymorph form, A of claim 1.

6. A pharmaceutical composition comprising a therapeutically effective amount of the ropinirole base substantially in polymorph form A of claim 2.

7. A pharmaceutical composition comprising a therapeutically effective amount of the ropinirole base substantially in polymorph form A of claim 3.

8. A process for the preparation of ropinirole base substantially in polymorph form A, the process comprising:
(a) treating crude ropinirole base or a salt thereof with an inorganic base in a solvent; and
(b) recovering the ropinirole substantially in polymorphic Form A.

9. The process of claim 8, wherein the solvent is selected from the group consisting of water, water-miscible solvent, water-immiscible solvent and mixtures thereof.

10. The process of claim 9, wherein the water-miscible solvent is selected from the group consisting of methanol, ethanol, isopropanol, t-butanol, acetonitrile, 1,4-dioxane and mixtures thereof.

11. The process of claim 9, wherein the water-immiscible solvent is selected from the group consisting of ethyl acetate, ethyl acetoacetate, methylene chloride, ethylene chloride, chloroform, methyl tert butyl ether, toluene and mixtures thereof.

12. The process of claim 5, wherein the inorganic base is selected from the group consisting of an aqueous or solid alkaline earth metal hydroxide, alkali or alkaline earth metal carbonate and mixtures thereof.

13. The process of claim 12, wherein the alkaline earth metal hydroxide is selected from the group consisting of sodium hydroxide, potassium hydroxide, barium hydroxide, calcium hydroxide and mixtures thereof.

14. The process of claim 12, wherein the alkali or alkaline earth metal carbonate is selected from the group consisting of sodium bicarbonate, potassium carbonate, calcium carbonate, barium carbonate and mixtures thereof.

15. Substantially pure ropinirole hydrochloride having less than about 0.15% weight of any one of 4-[2-(n-propylamino)ethyl]-1,3-dihydro-2H-indol-2-one, 4-(2-(dipropylamino)ethyl)-1-hydroxyindolin-2-one, and 4-ethyl-1,3-dihydro-2H-indol-2-one.

16. The substantially pure ropinirole hydrochloride of claim 15, having less than about 0.15% weight of each of 4-[2-(n-propylamino)ethyl]-1,3-dihydro-2H-indol-2-one, 4-(2-(dipropylamino)ethyl)-1-hydroxyindolin-2-one, and 4-ethyl-1,3-dihydro-2H-indol-2-one.

17. A pharmaceutical composition comprising a therapeutically effective amount of the substantially pure ropinirole hydrochloride of claim 15.

18. A process for the preparation of substantially pure ropinirole hydrochloride, the process comprising:
(a) providing a solution comprising ropinirole base of Formula II in one or more organic solvents; 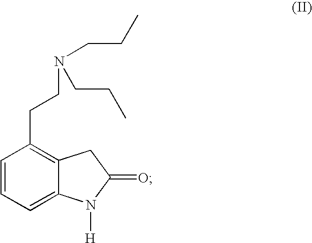
(b) extracting the solution with a base at a pH of about 10 to about 12;
(c) extracting the solution with an acid;
(d) substantially removing the solvent from the solution;
(e) dissolving the resulting solids in one or more polar solvents;
(f) adding hydrochloric acid under substantially dry conditions; and
(g) recovering the substantially pure ropinirole hydrochloride.

19. Ropinirole hydrochloride prepared according to the process of claim 18, having a purity of at least about 99.5% as determined by HPLC.
